(19) United States
(12) Patent Application Publication
(10) Pub. No.: US 2023/0092815 A1
(43) Pub. Date: Mar. 23, 2023

(54) COMPOSITION AND FUNCTIONAL FOOD PRODUCT COMPRISING GREEN TEA EXTRACT

(71) Applicants: Akari Nakasone, Miyoshi -shi, Aichi (JP); Madoka Abe, Nagoya-shi, Aichi (JP); Hirofumi Tachibana, Fukuoka-shi, Fukuoka (JP)

(72) Inventors: Akari Nakasone, Miyoshi -shi, Aichi (JP); Madoka Abe, Nagoya-shi, Aichi (JP); Hirofumi Tachibana, Fukuoka-shi, Fukuoka (JP)

(73) Assignees: Toyota Jidosha Kabushiki Kaisha, Toyota-shi, Aichi (JP); Kyushu University, National University Corporation, Fukuoka-shi Fukuoka (JP)

(21) Appl. No.: 17/908,811

(22) PCT Filed: Mar. 3, 2021
(86) PCT No.: PCT/JP2021/008183

§ 371 Date: Sep. 1, 2022

(30) Foreign Application Priority Data
Mar. 4, 2020 (JP) 2020-036833

(51) Int. Cl. A61K 36/82; A23L 33/105; A23F 3/16; A61K 36/752; A61K 31/7048; A61K 31/353

(52) U.S. Cl. CPC A61K 36/82; A23L 33/105; A23F 3/16; A61K 36/752; A61K 31/7048; A61K 31/353; A23V 2002/00

(57) ABSTRACT

The present disclosure is intended to enhance various effects of a green tea extract such as catechin in the most effective manner. The ratio (B/A) of epigallocatechin gallate (A) contained in the green tea extract and flavanone glycoside (B) contained in the citrus extract is 0.2<B/A<1.6, or a ratio (C/A) of epigallocatechin gallate (A) and eriocitrin (C) contained in the citrus extract is 0.2<C/A<0.5.

### CROSS-REFERENCE TO RELATED APPLICATIONS

The present application is a National Stage Entry under 35 U.S.C. § 371 of International Patent Application PCT/JP2021/008183, filed Mar. 3, 2021, which claims priority to Japanese Patent Application 2020-036833, filed Mar. 4, 2020, the contents of which are hereby incorporated by reference in their entirety.

### TECHNICAL FIELD

The present description relates to a composition, a functional food product, and the like comprising a green tea extract, such as catechin, and a citrus fruit extract or flavanone glycoside.

### BACKGROUND

In recent years, cancers account for one third of deaths in Japan, and there is therefore an urgent need to establish appropriate therapeutic means for cancer treatment. In multiple myeloma, the therapeutic environment is now being improved by introduction of lenalidomide and bortezomib (i.e., a specific inhibitor of proteasomes). On the other hand, there are numerous cases where cancer cells have acquired resistance to existing drugs, thereby causing recurrence. For this reason, there has been a need to develop an anticancer agent having a different action mechanisms from conventional drugs.

Further, such an anticancer agent can be used in combination with existing therapies if they differ in their dose limiting toxicity (DLT), which allows planning of a more effective therapeutic strategy.

EGCG (epigallocatechin gallate, epigallocatechin-O-gallate), which is one of the major catechins contained in green tea, has been reported to have an anticancer effect (Non-Patent Literature 1), and a phase II clinical trial has been conducted in patients with chronic lymphocytic leukemia, which is a type of blood cancer (Non-Patent Literature 2). While EGCG is known to exert an anticancer effect upon binding to its target molecule 67-kDa laminin receptor (67LR) on the cell membrane, the lethal effect of EGCG on leukemia cells or multiple myeloma cells is limited (Non-Patent Literature 2). Accordingly, there has been a strong demand for enhancement of the effects of EGCG when it is used as an anticancer agent.

Patent Literature 1 discloses that a citrus fruit extract or flavanone glycoside enhances an anti-cancer effect and other effects of catechin, a composition comprising a green tea extract such as catechin and a citrus fruit extract or flavanone glycoside has various effects, such as an anti-cancer effect, an anti-amyotrophic effect, and an anti-obesity effect.

### CITATION LIST

### Patent Literature

{PTL 1}

WO 2015/199169

### Non Patent Literature

{NPL 1}

Khan N, Afaq F, Saleem M, et al. Targeting multiple signaling pathways by green tea polyphenol (-)-epigallocatechin-3-gallate, Cancer. res., 2006; 66: 2500-2505

{NPL 2}

Shanafelt T D, Call T G, and Zent C S, et al., Phase I trial of daily oral Polyphenon E in patients with asymptomatic Rai stage 0 to II chronic lymphocytic leukemia, J. Clin. Oncol., 2009; 27: 3808-3814

### SUMMARY

### Technical Problem

While Patent Literature 1 suggests that a citrus fruit extract or flavanone glycoside would enhance various effects of a green tea extract such as catechin, an extent of enhancement was not sufficient. Accordingly, it is an object of embodiments of the present description to provide a composition and a functional food product that can enhance various effects of a green tea extract such as catechin, including an anti-cancer effect, in the most effective manner.

### Solution to Problem

The present inventors have conducted concentrated studies in order to address the problem described above. As a result, they discovered that various effects of a green tea extract, such as catechin, would be enhanced to a significant extent by mixing a green tea extract, such as catechin, with a citrus fruit extract or flavanone glycoside at a given ratio. This has led to the embodiments of the present description.

Embodiments of the present disclosure include the following.

(1) A green tea extract-containing composition comprising a green tea extract and a citrus fruit extract, wherein a ratio (B/A) of epigallocatechin gallate (A) contained in the green tea extract and flavanone glycoside (B) contained in the citrus extract is 0.2<B/A<1.6, or a ratio (C/A) of epigallocatechin gallate (A) and eriocitrin (C) contained in the citrus extract is 0.2<C/A<0.5.

(2) The green tea extract-containing composition according (1), wherein the ratio (B/A) is 0.4≤B/A≤0.8.

(3) The green tea extract-containing composition according to (1), wherein the ratio (C/A) is 0.25≤C/A≤0.34.

(4) The green tea extract-containing composition according to (1), wherein the flavanone glycoside is transglycosylated hesperidin.

(5) A functional food product comprising a green tea extract and a citrus fruit extract, wherein a ratio (B/A) of epigallocatechin gallate (A) contained in the green tea extract and flavanone glycoside (B) contained in the citrus extract is 0.2<B/A<1.6, or a ratio (C/A) of epigallocatechin gallate (A) and eriocitrin (C) contained in the citrus extract is 0.2<C/A<0.5.

(6) The functional food product according to (5), wherein the ratio (B/A) is 0.4≤B/A≤0.8.

(7) The functional food product according to (5), wherein the ratio (C/A) is 0.25≤C/A≤0.34.

(8) The functional food product according to (5), wherein the flavanone glycoside is transglycosylated hesperidin.

Embodiments of the present description also relate to an agent selected from the group consisting of an anti-cancer agent, an anti-muscle atrophy agent, an anti-obesity agent, an anti-inflammatory agent, a cholesterol-lowering agent, a prophylactic agent for thrombosis or cerebral infarction, and an immunostimulatory agent, comprising a green tea extract and a citrus fruit extract, wherein a ratio (B/A) of epigallocatechin gallate (A) contained in the green tea extract and flavanone glycoside (B) contained in the citrus extract is 0.2<B/A<1.6, or a ratio (C/A) of epigallocatechin gallate (A) and eriocitrin (C) contained in the citrus extract is 0.2<C/A<0.5.

In addition, embodiments of the present description relate to an enhancer, which enhances at least one effect of a green tea extract or catechin selected from the group consisting of an anti-cancer effect, an anti-amyotrophic effect, an anti-obesity effect, an anti-inflammatory effect, a cholesterol-lowering effect, a prophylactic effect on thrombosis or cerebral infarction, and an immunostimulatory effect, comprising a green tea extract and a citrus fruit extract, wherein a ratio (B/A) of epigallocatechin gallate (A) contained in the green tea extract and flavanone glycoside (B) contained in the citrus extract is 0.2<B/A<1.6, or a ratio (C/A) of epigallocatechin gallate (A) and eriocitrin (C) contained in the citrus extract is 0.2<C/A<0.5.

Further, embodiments of the present description relate to a method of administering a composition comprising a green tea extract and a citrus fruit extract, wherein a ratio (B/A) of epigallocatechin gallate (A) contained in the green tea extract and flavanone glycoside (B) contained in the citrus extract is 0.2<B/A<1.6, or a ratio (C/A) of epigallocatechin gallate (A) and eriocitrin (C) contained in the citrus extract is 0.2<C/A<0.5, to a subject, so as to enhance at least one effect of the green tea extract or catechin selected from the group consisting of an anti-cancer effect, an anti-amyotrophic effect, an anti-obesity effect, an anti-inflammatory effect, a cholesterol-lowering effect, a prophylactic effect on thrombosis or cerebral infarction, and an immunostimulatory effect in the subject (provided that medical practice on humans is excluded in some instances).

In embodiments of the present description, the green tea extract can be at least one catechin selected from the group consisting of epicatechin, epigallocatechin, epicatechin gallate, gallocatechin gallate, epigallocatechin gallate, and methylated catechin.

In embodiments of the present description, examples of citrus fruit extracts include flavanone glycoside, eriodictyol, and naringenin. An example of flavanone glycoside is transglycosylated hesperidin.

In an embodiment of the present description, a green tea extract is gallocatechin gallate, epigallocatechin gallate, or methylated catechin, and a citrus fruit extract is eriodictyol.

The composition, the food product, the agent, and the enhancer of embodiments of the present description have at least one effect selected from the group consisting of an anti-cancer effect, an anti-amyotrophic effect, an anti-obesity effect, an anti-inflammatory effect, a cholesterol-lowering effect, a prophylactic effect on thrombosis or cerebral infarction, and an immunostimulatory effect.

### Advantageous Effects

In the composition and the functional food product of embodiments of the present description, the ratio of epigallocatechin gallate in the green tea extract to flavanone glycoside in the citrus fruit extract is within a given range or the ratio of epigallocatechin gallate to eriocitrin is within a given range. Thus, various effects of epigallocatechin gallate are enhanced to a significant extent. This enables the composition and the functional food product of embodiments of the present disclosure to exert an excellent anti-cancer effect and other effects.

### BRIEF DESCRIPTION OF DRAWINGS

FIG. 1 is a characteristic diagram showing the results of measurement of plasma cGMP concentration upon administration of various compositions.

FIG. 2 is a characteristic diagram showing the results of measurement of plasma cGMP concentration upon administration of various compositions.

FIG. 3 is a characteristic diagram showing the results of measurement of plasma cGMP concentration upon administration of various compositions.

### DESCRIPTION OF EMBODIMENTS

The composition and the functional food product of embodiments of the present description each comprise a green tea extract and a citrus fruit extract, wherein a ratio (B/A) of epigallocatechin gallate (A) contained in the green tea extract and flavanone glycoside (B) contained in the citrus extract is 0.2<B/A<1.6, or a ratio (C/A) of epigallocatechin gallate (A) and eriocitrin (C) contained in the citrus extract is 0.2<C/A<0.5.

As disclosed in WO 2015/199169, a citrus fruit extract comprising eriodictyol, which is a polyphenol, has an effect of enhancing various effects of a green tea extract, such as epigallocatechin gallate (epigallocatechin-O-gallate (EGCG)), including an anti-cancer effect. Examples of effects of a green tea extract include an anti-obesity effect, an anti-inflammatory effect, a cholesterol-lowering effect, an antithrombotic effect, an immunostimulatory effect, and an anti-amyotrophic effect (WO 2015/199169). A green tea extract is reported to have an anti-cancer effect, an anti-insulin resistance effect, an anti-inflammatory effect, an antiallergic effect, an anti-amyotrophic effect, a prophylactic effect on arteriosclerosis, an antithrombotic effect, or a prophylactic effect on Alzheimer's disease.

WO 2015/199169 describes as follows. That is, use of (a1) eriodictyol or a structural analog thereof, i.e., naringin or hesperidin, (a2) glycosides of these polyphenols which may be metabolized in vivo as these polyphenols, or (a3) food products containing (a1) or (a2) in combination with (b1) EGCG, (b2) methylated EGCG which also serves as a 67LR agonist, as in the case of EGCG, or (b3) food products containing (b1) or (b2) exerts an anticancer effect, an anti-amyotrophic effect, an anti-obesity effect, an anti-insulin resistance effect, an anti-inflammatory effect, an antiallergic effect, a prophylactic effect on arteriosclerosis, an antithrombotic effect, an anti-neurodegenerative effect, and an anti-inflammatory effect. Accordingly, the above combinations between (a1), (a2), or (a3) and (b1), (b2), or (b3) are useful as food products, medicaments, or supplements intended for prevention or treatment achieved by the effects as described above of diseases, such as thrombotic diseases (e.g., pulmonary embolism, DIC, myocardial infarction, or cerebral infarction), cancers, amyotrophy, obesity, insulin resistance diseases, inflammatory diseases (e.g., Sjogren's disease and collagenosis), allergic diseases, arteriosclerosis, and neurodegenerative diseases (e.g., brain diseases such as Alzheimer's disease and dementia).

(1) Green Tea Extract

The green tea extract is prepared from tea plant, which is an evergreen tree of the family Theaceae, and such extract contains at least epigallocatechin gallate. Examples of green tea plants include tea plants, such as Camellia taliensis and Camellia sinensis. Examples of green tea varieties that can be used include tea plant (Camellia sinensis (L.) Kuntze), Assam tea plant (Camellia sinensis (L.) Kuntze var assamica (J. W. Mast) Kitam.), hybrids between Camellia sinensis and Camellia taliensis, tea varieties “Yabukita,” “Benifuuki,” “Benifuji,” “Benihomare,” “Yaeho,” “Surugawase,” “Yutakamidori,” “Kanayamidori,” “Okumusashi,” “Seisindaipan,” “Seisin oolong,” “Ohba oolong,” “Benibana,” “Benihikari,” “Yamakai,” “Yamamidori,” “Karabeni,” “Koushun,” “Soufuu,” “Fukumidori,” “Minekaori,” “Benihikari,” “Minamikaori,” “Izumi,” “Fuushun,” “Tamamidori,” “Kuritawase,” “Shunmei,” “Sayamamidori,” “Asagiri,” “Hokumei,” “Tadanishiki,” “Asahi,” “Sayamakaori,” “Meiryoku,” “Yamatomidori,” “Asatsuyu,” “Toyoka,” “Natsumidori,” “Ujihikari,” “Ooiwase,” “Gokoh,” “Inzatsu 131,” “Makinoharawase,” “Takachiho,” “Komakage,” “Samidori,” “Komakage,” “Hatsumomiji,” “Ryoufuu,” “Minamisayaka,” “Saemidori,” “Okuyutaka,” “Fujimidori,” “Sunrouge,” and “Okumidori.” Among them, “Yabukita,” “Benifuuki,” “Kanayamidori,” “Okumusashi,” “Soufuu,” “Fuushun,” “Tadanishiki,” and “Sunrouge” are used in some embodiments. In addition, examples of tea leaves from these tea plants include green tea, refined green tea, coarse green tea, twig green tea, bud green tea, brown rice green tea, broken green tea, powdered green tea, parched green tea, Chinese sweet tea, Pouchong tea, oolong tea, and black tea.

While an extraction solvent is not particularly limited, water, an organic solvent, or a mixture of water and an organic solvent may be used.

Examples of an organic solvent include polar organic solvents, such as lower alcohols containing 1 to 4 carbon atoms (e.g., methanol, ethanol, propanol, isopropanol, n-butanol, isobutanol, sec-butanol, and tert-butanol) and ketones (e.g., dimethyl ketone, methyl ethyl ketone, acetone, and methyl isobutyl ketone), and nonpolar organic solvents, such as methyl acetate, ethyl acetate, butyl acetate, and diethyl ether. It is also possible to use any of these polar organic solvents in adequate combination with any of these nonpolar organic solvents. Hot water, ethanol, and hydrous ethanol are used in some embodiments. The alcohol concentration in hydrous alcohols is 30 v/v % to 90 v/v % in embodiments, and 40 v/v % to 70 v/v % in embodiments. In the case of hot water, its temperature is 40 degrees C. to 100 degrees C. in embodiments, and 60 degrees C. to 100 degrees C. in embodiments.

Examples of extraction techniques to obtain a green tea extract include conventional techniques, such as immersion extraction, heating extraction, continuous extraction, and supercritical extraction. The green tea extract thus obtained may then be concentrated in accordance with a conventional technique. The resulting green tea extract, concentrate, or the like may further be purified in accordance with a conventional technique. Examples of purification techniques include ultrafiltration, treatment with adsorbent resins, molecular chromatography, partition chromatography, and liquid-liquid extraction.

Examples of drying techniques include, but are not limited to, spray drying and freeze drying. A green tea extract may contain polyphenols, catechins, and the like, in addition to epigallocatechin gallate. In embodiments, a green tea extract contains catechin, epicatechin, epigallocatechin, catechin gallate, epicatechin gallate, gallocatechin gallate, methylated catechin, and the like.

Major members of methylated catechin intended in embodiments of the present disclosure include epigallocatechin-3-O-(3-O-methyl)gallate (hereafter, referred to as “EGCG 3″Me”), epicatechin-3-O-(3-O-methyl)gallate (hereafter, referred to as “ECG 3″Me”), epicatechin-3-O-(4-O-methyl)gallate (hereafter, referred to as “ECG 4″Me”), epigallocatechin-3-O-(4-O-methyl)gallate (hereafter, referred to as “EGCG 4″Me”), gallocatechin-3-O-(3-O-methyl)gallate (hereafter, referred to as “GCG 3″Me”), catechin-3-O-(3-O-methyl)gallate (hereafter, referred to as “CG 3″Me”), catechin-3-O-(4-O-methyl)gallate (hereafter, referred to as “CG 4″Me”), or gallocatechin-3-O-(4-O-methyl)gallate (hereafter, referred to as “GCG 4″Me”), and isomerized products thereof.

The content of a green tea extract in the composition will vary depending on the dosage form of the composition or the mode of its administration, but it may be adequately determined in consideration of the content of a citrus fruit extract described below. In embodiments of the present disclosure, moreover, the composition may comprise another catechin different from the catechins contained in a green tea extract. An example thereof is synthetic catechins. Synthetic catechins may be obtained in accordance with a conventional technique (Chem. Asian J. 2010, 5, 2231-2248. DOI: 10.1002/asia.201000372).

A commercially available green tea extract may also be used. An example of a commercially available green tea extract that can be used is POLYPHENON (registered trademark) manufactured by Mitsui Norin Co., Ltd.

(2) Citrus Fruit Extract

The citrus fruit extract is a product extracted from a citrus fruit, which contains at least eriocitrin or flavanone glycoside. Examples of a citrus fruit include the following.

Examples of the genus Citrus include orange, grapefruit, Citrus junos, bitter orange, Citrus sphaerocarpa, Citrus sudachi, Citrus yuko hort.ex Tanaka, Yukou (a native Japanese citrus), Citrus depressa, lemon, lime, Citrus natsudaidai, Citrus hassaku, Citrus iyo, Citrus grandis, mandarin orange, satsuma mandarin, Cirus reticulata, Citrus tachibana, Citrus kinokuni, Valencia orange, navel orange, blood orange, Jaffa orange, bergamot orange, and Chinotto orange.

In addition to the above citrus fruits of the genus Citrus, trifoliate oranges, kumquats and others may also be used.

As an extraction solvent used to extract a citrus fruit, water, an organic solvent, or a mixture of water and an organic solvent may be used, as described above. Examples of an organic solvent include polar organic solvents, such as lower alcohols containing 1 to 4 carbon atoms (e.g., methanol, ethanol, propanol, isopropanol, n-butanol, isobutanol, sec-butanol, and tert-butanol) and ketones (e.g., dimethyl ketone, methyl ethyl ketone, acetone, and methyl isobutyl ketone), and nonpolar organic solvents, such as methyl acetate, ethyl acetate, butyl acetate, and diethyl ether. Water or ethanol is used in some embodiments. It is also possible to use any of these polar organic solvents in adequate combination with any of these nonpolar organic solvents.

Examples of extraction techniques to obtain a citrus fruit extract include conventional techniques, such as immersion extraction, heating extraction, continuous extraction, and supercritical extraction. The extract may then be concentrated in accordance with a conventional technique. The resulting citrus fruit extract, concentrate, or the like may further be purified in accordance with a conventional technique. Examples of purification techniques include ultrafiltration, treatment with adsorbent resins, molecular chromatography, partition chromatography, and liquid-liquid extraction.

A citrus fruit extract contains at least eriocitrin or flavanone glycoside. Examples of flavanone glycoside include, but are not particularly limited to, hesperidin, naringin, poncirin, and sakuranin. A citrus fruit extract may further contain flavanones, such as butyne, eriodictyol, hesperetin, homoeriodictyol, isosakuranetin, naringenin, pinocembrin, sakuranetin, or sterubin, in addition to eriocitrin or flavanone glycoside.

The composition may comprise another flavanone or flavanone glycoside different from the flavanone or flavanone glycoside contained in a citrus fruit extract. Examples include synthetic flavanone and a transglycosylated compound of flavanone comprising a sugar molecule bound thereto, such as transglycosylated hesperidin. Specific examples include synthetic eriodictyol, synthetic naringenin, and synthetic hesperetin, which may be used alone or in combinations of two or more. Synthetic eriodictyol, synthetic naringenin, and synthetic hesperetin may be obtained in accordance with a conventional technique (European J. Org. Chem., 2012 (3): 449-462, doi:10.1002/ejoc.201101228). An example of transglycosylated hesperidin that can be used is transglycosylated hesperidin available from Hayashibara Co., Ltd. and Glico Nutrition Co., Ltd.

(3) Composition

In a composition of the present disclosure, a ratio (B/A) of epigallocatechin gallate (A) contained in the green tea extract and flavanone glycoside (B) contained in the citrus extract is 0.2<B/A<1.6, or a ratio (C/A) of epigallocatechin gallate (A) and eriocitrin (C) contained in the citrus extract is 0.2<C/A<0.5. In some embodiments, the ratio (B/A) is 0.4≤B/A≤0.8, and in some embodiments 0.5≤B/A≤0.7. in some embodiments, the ratio (C/A) is 0.25≤C/A≤0.34, and in some embodiments C/A=0.3.

By maintaining the ratios mentioned above, various effects of epigallocatechin gallate, such as an anti-cancer effect, can be enhanced to a significant extent. Various effects of epigallocatechin gallate can be determined based on the amount of cyclic guanosine monophosphate (cGMP) production. It has been found that epigallocatechin gallate activates endothelial NO synthase (eNOS) via 67LR to induce NO production and then accelerates cGMP production in a soluble guanylate cyclase (sGC)-dependent manner. In addition, it has been found that cGMP activates protein kinase C-delta (PKC-delta) and acidic sphingomyelinase (ASM). Specifically, cGMP is associated with a signal pathway through which epigallocatechin gallate induces apoptosis in cancer cells and cGMP serves as a biomarker for various effects of epigallocatechin gallate.

In addition to the ingredients described above, the composition may further comprise a carrier acceptable for use in food products and other known or well-known additives. Examples of the additives include those commonly used in medicaments or food products, such as excipients, binders, lubricants, disintegrators, coloring agents, correctives, emulsifiers, surfactants, solubilizers, suspending agents, isotonizing agents, buffering agents, antiseptics, antioxidants, stabilizers, and absorbefacients, which may be used in adequate combination, according to need.

The composition may be in any of liquid, solid, powder, and gel forms, and the composition may be formulated into any oral dosage form, such as tablets, powders, capsules (hard capsules or soft capsules), granules, pills, solutions, or syrups. These formulations may be prepared in accordance with a conventional technique. When the composition is in a solution form, water and other aqueous media can be used as carriers in some embodiments.

When the composition is in a solid form, ingredients to be added include excipients, such as crystalline cellulose, magnesium stearate, and calcium stearate, and expanders, such as corn starch and alginic acid.

Examples of compounds required for formulation into a powder, solid, or solution dosage form include erythritol, maltitol, hydroxypropyl cellulose, kaolin, and talc.

The composition has at least one effect selected from the group consisting of an anti-cancer effect, an anti-amyotrophic effect, an anti-obesity effect, an anti-inflammatory effect, a cholesterol-lowering effect, a prophylactic effect on thrombosis or cerebral infarction, and an immunostimulatory effect. Thus, the above composition can be used as an anticancer agent, an anti-amyotrophic agent, an anti-obesity agent, an anti-inflammatory agent, a cholesterol-lowering agent, a prophylactic agent for thrombosis or cerebral infarction, or an immunostimulatory agent. In the above respective agents, the method for preparing a tea extract or a catechin and a citrus fruit extract or a flavanone and the content of these respective ingredients are as described above.

Examples of subjects to be fed with the composition of embodiments of the present disclosure include, but are not particularly limited to, humans, non-human mammals, such as laboratory animals (e.g., mice, rats, guinea pigs, and rabbits), domestic animals (e.g., cows, horses, pigs, and goats), and pet animals (e.g., dogs, cats and other pets). The composition of embodiments of the present disclosure can be expected to prevent or treat cancers, amyotrophy (e.g., amyotrophic lateral sclerosis (ALS)), inflammatory diseases, thrombosis or cerebral infarction, hyperlipidemia, and infections, or to improve lifestyle-related diseases and obesity.

The amount of the composition of embodiments of the present disclosure to be fed per kg of the body weight is, in terms of the amount of epigallocatechin gallate, 0.1 to 30 mg per day in some embodiments, 0.1 to 20 mg per day in some embodiments, 0.1 to 10 mg per day in some embodiments, and 0.1 to 5 mg per day in some embodiments. In the composition of embodiments of the present disclosure, the ratio (B/A) of epigallocatechin gallate (A) and flavanone glycoside (B), or the ratio (C/A) of epigallocatechin gallate (A) and eriocitrin (C) is within a given range. At the low dose of epigallocatechin gallate as described above, accordingly, the various effects achieved by epigallocatechin gallate can be sufficiently exerted.

(4) Food Product

The food product of embodiments of the present disclosure comprises a green tea extract and a citrus fruit extract, in which a ratio (B/A) of epigallocatechin gallate (A) contained in the green tea extract and flavanone glycoside (B) contained in the citrus extract is 0.2<B/A<1.6, or a ratio (C/A) of epigallocatechin gallate (A) and eriocitrin (C) contained in the citrus extract is 0.2<C/A<0.5. Since the food product contains such ingredients at a given ratio, in particular, it can be used as a functional food product, a supplement, or the like intended to exert at least one effect selected from the group consisting of an anticancer effect, an anti-amyotrophic effect, an anti-obesity effect, an anti-inflammatory effect, a cholesterol-lowering effect, a prophylactic effect on thrombosis or cerebral infarction, and an immunostimulatory effect.

The food products (functional food products, in particular) of embodiments of the present disclosure may be in any form, such as supplements (i.e., powders, granules, soft capsules, hard capsules, tablets, chewable tablets, or rapidly disintegrating tablets), beverages (e.g., tea beverages, carbonated beverages, lactic acid beverages, or sports drinks), confectioneries (e.g., gums, chocolates or cookies, candies), oils, edible fat and oil products (e.g., mayonnaise, dressings, or butter), seasonings (e.g., ketchups or sauces), fluid diets, dairy products (e.g., cow milk, yogurt, or cheese), bakery products, or noodles (e.g., white wheat noodles, buckwheat noodles, Chinese noodles, pasta, Hiyamugi (Japanese vermicelli), or rice vermicelli). It should be noted that the food product of embodiments of the present disclosure is not limited to these forms.

Examples of subjects to be fed with the composition of embodiments of the present disclosure include, but are not particularly limited to, humans, non-human mammals, such as laboratory animals (e.g., mice, rats, guinea pigs, and rabbits), domestic animals (e.g., cows, horses, pigs, and goats), and pet animals (e.g., dogs, cats and other pets).

The amount of the food product of embodiments of the present disclosure to be fed per kg of the body weight is, in terms of the amount of epigallocatechin gallate, 0.1 to 30 mg per day in some embodiments, 0.1 to 20 mg per day in some embodiments, 0.1 to 10 mg in some embodiments, and 0.1 to 5 mg per day in some embodiments. In the food product of embodiments of the present disclosure, the ratio (B/A) of epigallocatechin gallate (A) and flavanone glycoside (B), or the ratio (C/A) of epigallocatechin gallate (A) and eriocitrin (C) is within a given range. At the low dose of epigallocatechin gallate as described above, accordingly, the various effects achieved by epigallocatechin gallate can be sufficiently exerted.

(5) Enhancer

The enhancer comprises a green tea extract and a citrus fruit extract in a manner such that a ratio (B/A) of epigallocatechin gallate (A) contained in the green tea extract and flavanone glycoside (B) contained in the citrus extract is 0.2<B/A<1.6, or a ratio (C/A) of epigallocatechin gallate (A) and eriocitrin (C) contained in the citrus extract is 0.2<C/A<0.5. Thus, various effects of epigallocatechin gallate, such as an anti-cancer effect, an anti-obesity effect, an anti-inflammatory effect, a cholesterol-lowering effect, an antithrombotic effect, an immunostimulatory effect, and an anti-amyotrophic effect, are enhanced. In an epigallocatechin gallate enhancer comprising flavanone glycoside (B) or eriocitrin (C) in the citrus fruit extract, specifically, the ratio of flavanone glycoside (B) or eriocitrin (C) to epigallocatechin gallate (A) is 0.2<B/A<1.6 or 0.2<C/A<0.5. In other words, the epigallocatechin gallate enhancer enhances at least one effect of epigallocatechin gallate selected from the group consisting of an anti-cancer effect, an anti-amyotrophic effect, an anti-obesity effect, an anti-inflammatory effect, a cholesterol-lowering effect, a prophylactic effect on thrombosis or cerebral infarction, and an immunostimulatory effect.

A subject is fed with a green tea extract and a citrus fruit extract in a manner such that a ratio (B/A) of epigallocatechin gallate (A) contained in the green tea extract and flavanone glycoside (B) contained in the citrus extract would be 0.2<B/A<1.6, or a ratio (C/A) of epigallocatechin gallate (A) and eriocitrin (C) contained in the citrus extract would be 0.2<C/A<0.5. Thus, at least one effect selected from the group consisting of an anticancer effect, an anti-amyotrophic effect, an anti-obesity effect, an anti-inflammatory effect, a cholesterol-lowering effect, a prophylactic effect on thrombosis or cerebral infarction, and an immunostimulatory effect of epigallocatechin gallate can be enhanced in the subject. It should be noted that medical practice on humans can be excluded.

Examples of subjects to be fed with the enhancer or subjected to the method of enhancement are as described above. Examples thereof include, but are not particularly limited to, humans, non-human mammals, such as laboratory animals (e.g., mice, rats, guinea pigs, and rabbits), domestic animals (e.g., cows, horses, pigs, and goats), and pet animals (e.g., dogs, cats and other pets).

With the use of the enhancer of embodiments of the present disclosure, the amount of epigallocatechin gallate to be fed per kg of the body weight can be 0.1 to 30 mg per day in some embodiments, 0.1 to 20 mg per day in some embodiments, 0.1 to 10 mg per day in some embodiments, and 0.1 to 5 mg per day in some embodiments. In the enhancer of embodiments of the present disclosure, the ratio (B/A) of epigallocatechin gallate (A) and flavanone glycoside (B), or the ratio (C/A) of epigallocatechin gallate (A) and eriocitrin (C) is within a given range. At the low dose of epigallocatechin gallate as described above, accordingly, the various effects achieved by epigallocatechin gallate can be sufficiently exerted.

### EXAMPLES

Hereafter, embodiments of the present disclosure is described in greater detail with reference to the examples, although the technical scope of the present disclosure is not limited to the following examples.

### Example 1

### Administration Example 1

EGCG (Sigma-Aldrich) and various flavonoids (eriocitrin: ALB Technology; transglycosylated hesperidin: Hayashibara Co., Ltd.; or eriodictyol: EXTRASYNTHESE) were dissolved in pure water, and the resulting solutions were orally administered to male C57BL/6J mice (6-week-old, body weight: 25 g, each group consisting of 3 or 6 mice) (Kyudo Co., Ltd.) using feeding tubes.

The mice were sacrificed by exsanguination through the aorta under anesthesia with isoflurane (with the addition of EDTA; final concentration: 1.5 mg/ml) 120 minutes after administration. The collected blood was centrifuged at 4 degrees C. and 200 times g for 15 minutes to collect plasma samples. The cGMP concentration in the plasma samples was measured using the TR-FRET kit (cisbio) and a fluorescent plate reader (EnVision (trademark) Multilabel Reader, PerkinElmer). Statistical processing was carried out using Statcel 4.0 (Excel admin software) by the Dunnett's test under a risk of 5% as statistically significant point.

### Administration Example 2

The green tea extract (containing 146 mg of EGCG per g) and transglycosylated hesperidin (alpha hesperidin PA-T, Glico Nutrition Co., Ltd.) were dissolved in pure water, and the resulting solutions were orally administered to male C57BL/6J mice (6-week-old, body weight: 25 g, each group consisting of 5 or 6 mice) (Kyudo Co., Ltd.) using feeding tubes.

The mice were sacrificed by exsanguination through the aorta under anesthesia with isoflurane (with the addition of EDTA; final concentration: 1.5 mg/ml) 120 minutes after administration. The collected blood was centrifuged at 4 degrees C. and 200 times g for 15 minutes to collect plasma samples. The cGMP concentration in the plasma samples was measured using the TR-FRET kit (cisbio) and a fluorescent plate reader (EnVision (trademark) Multilabel Reader, PerkinElmer). Statistical processing was carried out by the Student's t-test under a risk of 5% as statistically significant point.

### Results

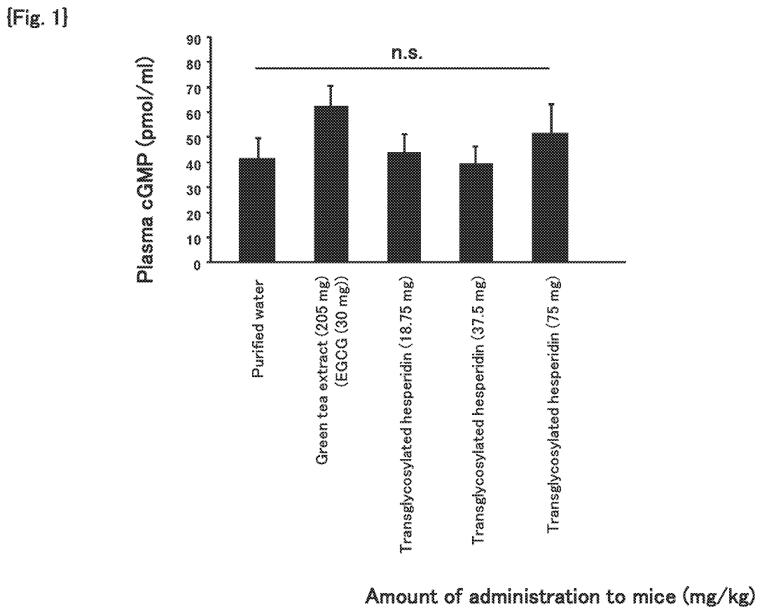

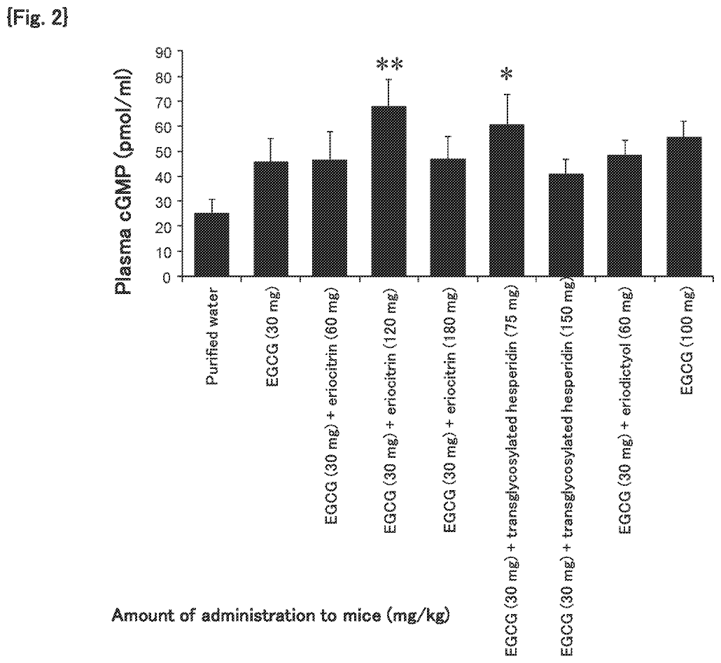

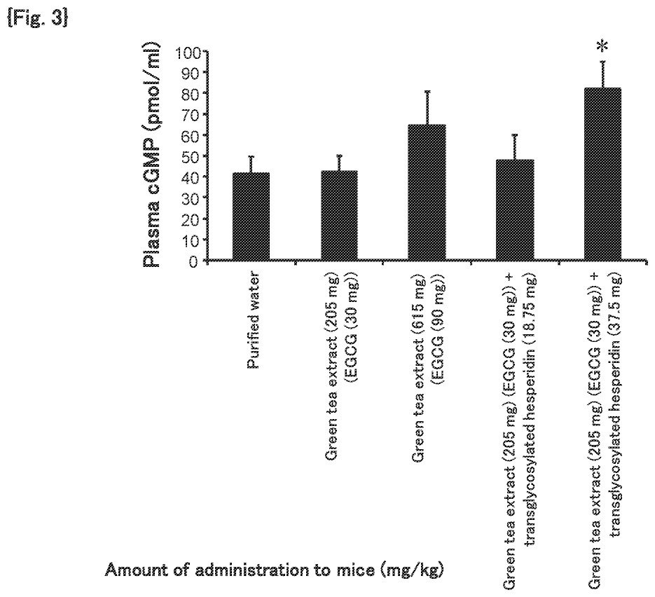

For comparison, only the green tea extract was administered to a group of mice, and transglycosylated hesperidin was administered to each of groups of mice at various levels in accordance with the method described in Administration Example 2. The results of measurement of cGMP concentrations of each group are shown in FIG. 1. The results of experiment of Administration Example 1 and those of Administration Example 2 are shown in FIG. 2 and FIG. 3, respectively. The amount of administration shown in FIGS. 1 to 3 is based on kg of mouse body weight (the amount administered to mice (mg/kg)).

Table 1 shows a summary of the results of experiments shown in FIG. 1 to FIG. 3. In columns indicating the results shown in Table 1, test groups exhibiting a significant difference in FIG. 2 and FIG. 3 are indicated as a circle.

TABLE 1
Summary of the amount of administration to mice/mg/kg

| | | | | Transglycosylated | | |
|---|---|---|---|---|---|---|
| | EGCG (A) | | | hesperidin (B) | | |
| EGCG (A) | 30.0 | 90.0 | 100.0 | | | |
| Eriocitrin (C) | | | | | | |
| Transglyocylated | | | | 18.8 | 37.5 | 75.0 |
| hesperididin (B) | | | | | | |
| Eriodictyol | | | | | | |
| Effects | X | | X | X | X | X |
| Ratio (A/B) or | | | | | | |
| (A/C) | | | | | | |

| | EGCG(A) + | | | | | | | |
|---|---|---|---|---|---|---|---|---|
| | transglycosylated | | | | EGCG (A) + | | | EGCG + |
| | hesperidin (B) | | | | eriocitrin | | | eriodictoyl |
| EGCG (A) | 30.0 | 30.0 | 30.0 | 30.0 | 30.0 | 30.0 | 30.0 | 30.0 |
| Eriocitrin (C) | | | | | 60.0 | 120.0 | 180.0 | |
| Transglyocylated | 18.8 | 37.5 | 75.0 | 150.0 | | | | |
| hesperididin (B) | | | | | | | | |
| Eriodictyol | | | | | | | | 60.0 |
| Effects | X | ◯ | ◯ | X | X | ◯ | X | X |
| Ratio (A/B) or | 1.6 | 0.80 | 0.4 | 0.20 | 0.5 | 0.3 | 0.2 | |
| (A/C) | | | | | | | | |

As shown in FIGS. 1 to 3 and Table 1, when the ratio (B/A) of epigallocatechin gallate (A) contained in the green tea extract and flavanone glycoside (B) contained in the citrus extract is 0.2<B/A<1.6, or the ratio (C/A) of epigallocatechin gallate (A) and eriocitrin (C) contained in the citrus extract is 0.2<C/A<0.5, the plasma cGMP concentration is increased to a significant extent. The results demonstrate that a composition comprising a green tea extract and a citrus fruit extract in which the ratio (B/A) of epigallocatechin gallate (A) contained in the green tea extract and flavanone glycoside (B) contained in the citrus extract is 0.2<B/A<1.6, or the ratio (C/A) of epigallocatechin gallate (A) and eriocitrin (C) contained in the citrus extract is 0.2<C/A<0.5 can enhance various effects of epigallocatechin gallate, such as an anti-cancer effect, to a significant extent.

1. A green tea extract-containing composition comprising a green tea extract and a citrus fruit extract, wherein a ratio (B/A) of epigallocatechin gallate (A) contained in the green tea extract and flavanone glycoside (B) contained in the citrus extract is 0.2<B/A<1.6, or a ratio (C/A) of epigallocatechin gallate (A) and eriocitrin (C) contained in the citrus extract is 0.2<C/A<0.5.

2. The green tea extract-containing composition according to claim 1, wherein the ratio (B/A) is 0.4≤A≤0.8.

3. The green tea extract-containing composition according to claim 1, wherein the ratio (C/A) is 0.25≤C/A≤0.34.

4. The green tea extract-containing composition according to claim 1, wherein the flavanone glycoside is transglycosylated hesperidin.

5. A functional food product comprising a green tea extract and a citrus fruit extract, wherein a ratio (B/A) of epigallocatechin gallate (A) contained in the green tea extract and flavanone glycoside (B) contained in the citrus extract is 0.2<B/A<1.6, or a ratio (C/A) of epigallocatechin gallate (A) and eriocitrin (C) contained in the citrus extract is 0.2<C/A<0.5.

6. The functional food product according to claim 5, wherein the ratio (B/A) is 0.4≤B/A≤0.8.

7. The functional food product according to claim 5, wherein the ratio (C/A) is 0.25≤C/A≤0.34.

8. The functional food product according to claim 5, wherein the flavanone glycoside is transglycosylated hesperidin.
